(19) United States
(12) Patent Application Publication
(10) Pub. No.: US 2009/0053771 A1
(43) Pub. Date: Feb. 26, 2009

(54) Process for making fuels and chemicals from AFEX-treated whole grain or whole plants

(75) Inventors: Bruce Dale, Mason, MI (US); Venkatesh Balan, East Lansing, MI (US); Shishir Chundawat, East Lansing, MI (US); Bryan Bals, South Bend, IN (US)

Correspondence Address: Ian C. McLeod;IAN C. McLEOD, P.C., 2190 Commons Parkway, Okemos, MI, 48864, US

(73) Assignee: Board of Trustees of Michigan State University, East Lansing, MI (US)

(21) Appl. No.: 12/229,225

(22) Filed: Aug. 21, 2008

Related U.S. Application Data
(60) Provisional application No. 60/965,735, filed on Aug. 22, 2007.

(51) Int. Cl. C12P 19/00; C12P 7/06

(52) U.S. Cl. 435/72; 435/161

(57) ABSTRACT

A process for hydrolyzing whole grain or whole plant biomass after an Ammonia Fiber Explosion (AFEX) process step is described. The process preferably uses a biomass that is hydrolyzed using a different combination of enzymes (amylase, cellulase and hemicellulase) to sugars for fermentation to produce ethanol. Harvesting the whole plant inclusive of grains and stalk for ethanol bio-processing is an economical route for future biorefineries. In addition to sugars, various value-added products like proteins and oil can be co-generated.

### CROSS REFERENCE TO RELATED APPLICATIONS

Priority to U.S. Provisional Application Ser. No. 60/965,735, filed Aug. 22, 2007, which is incorporated herein by reference in its entirety, is claimed.

### BACKGROUND OF THE DISCLOSURE

1. Field of the Disclosure

The disclosure generally relates to the use of ammonia to prepare biomass mixtures of lignocellulosic materials and grain (e.g., starch and cellulose/hemicellulose mixtures) for subsequent conversion to sugars by pretreating the mixed biomass at moderate temperatures and pressures. After removing most of the ammonia, the treated biomass is hydrolyzed by a combination of enzymes to produce simple sugars such as glucose and xylose.

2. Brief Description of Related Technology

A wide variety of methods (e.g., concentrated or dilute acids or bases, high temperatures, radiation in various forms) have been used to pretreat lignocellulosic biomass to increase the yield of sugars for many different uses. The goal of these pretreatments is to increase the rate and/or yield at which these sugars are obtained by chemical or biochemical means such as acid catalysis, enzymatic catalysis, fermentation or animal digestion. In general, these pretreatments have fallen short of desired economic and technical performance for several main reasons: (1) many pretreatments degrade some of the sugars, e.g. to acids or aldehydes, thus reducing yields and inhibiting subsequent biological conversion of the remaining sugars; (2) when chemicals are used to pretreat, it is frequently difficult to recover these chemicals at reasonable cost; (3) residual pretreatment chemicals can negatively affect downstream conversion operations; and (4) the effectiveness of many pretreatments is limited so that the ultimate conversions of structural carbohydrates obtained, independent of lost yield by sugar degradation reactions, is inadequate for competitive process economics. Thus, there are many “old” pretreatment methods, and they have numerous drawbacks.

Similarly, many treatments have been used to increase the rate and extent of conversion of starchy materials (e.g., corn and other grains) to fermentable sugars or more digestible starches. Usually, these treatments involve some combination of steam, heat and/or pressure to gelatinize starch, in some cases followed by digestion with starch hydrolyzing enzymes. Previously applied successful technologies do not simultaneously increase the rate and extent of both starch and lignocellulose conversion to sugars. Instead, previous technologies have been focused on either starch or lignocellulose conversion, but not both.

The United States' ethanol market as a fuel additive has been growing rapidly in recent years due to high oil prices and concerns over the environmental impact of MTBE as an oxygenate, another common gasoline additive. Approximately, 4 billion gallons of ethanol was produced in 2005, an increase of 17% over the previous year and over twice as much as 2001. This rapid growth is expected to continue, as 29 new ethanol plants are under construction to add to the 95 plants operating as of January 2006 (Renewable Fuels Association, 2006). The Energy Policy Act, signed into law in August 2005, requires that 7.5 billion gallons per year of biofuels, most notably ethanol, are to be mixed with gasoline by 2012, with several states adopting more stringent standards. In addition, ethanol is seen as an attractive alternative to oil as a fuel source, due to being renewable, environmentally friendly, and domestically produced. The U.S. Department of Energy (Energy Policy Acts, 2005) has stated its goal to replace 30% of U.S. gasoline demand with ethanol by 2030.

Currently, ethanol in the United States is made primarily from the starch in corn grain. The dominant procedure is the dry-grind process. The corn is ground and added to water to form a mash, which is then heated to release the starch. Amylase enzymes then hydrolyze the starch into glucose, and the resulting slurry is fermented to form ethanol. The solution is then distilled to recover the ethanol, which is purified using a molecular sieve (Bothast and Schlicher, 2004). However, grain alone can not meet the future demands for ethanol, as 13% of the total corn produced in the United States is already used for ethanol production (Renewable Fuels Association, 2006). Thus, a considerable amount of research is underway to produce ethanol from the cellulose and hemicellulose in plants, which are far more abundant in nature and cheaper to produce (Sun and Cheng, 2002; Knauf and Moniruzzaman, 2004; Gray et al., 2006).

However, enzymatic hydrolysis of cellulosic biomass generally results in low sugar yields unless the biomass undergoes a pretreatment step (Zhang and Lynd, 2004). A novel pretreatment method to improve the efficiency of the hydrolysis is the ammonia fiber explosion (AFEX) process (U.S. Pat. Nos. 4,600,590 and 6,106,888). Concentrated ammonia is added to the biomass under high pressure (200-500 psi) and moderate temperatures (60-200° C.) before rapidly releasing the pressure to destructure the plant material. This process compares favorably economically to other leading pretreatment methods, and continued research has further improved its economics (Eggeman and Elander, 2005). AFEX decrystallizes cellulose, hydrolyzes hemicellulose, removes and depolymerizes lignin, and greatly increases the overall porosity of the biomass, thereby significantly increasing the rate of enzymatic hydrolysis (Mosier et al., 2004). Previous work has shown this process to give near theoretical yields of glucose on different types of agricultural waste (Sulbaran-de-Ferrer et al., 2003, Teymouri et al., 2005).

It is likely that there will be a long transition period as society moves towards cellulosic ethanol. One likely outcome is that current ethanol plants can be refitted or expanded to produce ethanol from cellulosic materials in addition to the grains (Kamm and Kamm, 2004; Shah, 2003; Ohara, 2003). The most likely source of cellulosic material would be the stover or straw left from the grain harvest, as well as the fiber within the grain itself.

3. Objects

It is an object of the disclosure to use combinations of (1) anhydrous ammonia or concentrated ammonia/water mixtures and (2) elevated temperatures to increase the conversion of materials containing starch, cellulose and hemicellulose to more digestible products, either by animals or enzymes. Further, it is an object to combine starch and cellulosic processing. Thus, rather than separating the grain from the stover as in FIG. 1A, the entire plant (stover and grains) can be harvested at the same time and processed as one unit, as seen in FIG. 1B. This reduces the number of steps required to fully process the plant, simplifying the process and reducing the cost of production as well. In addition, it is an object to use the AFEX pretreatment which increases the susceptibility of both starch and cellulose present in whole grains to enzymatic hydrolysis, reducing the cost of hydrolysis, compared to the current dry grind process as seen in FIGS. 1C and 1D. In addition, it is an object to produce free sugars, and various value-added products like proteins and oil which could be co-generated.

These and other objects may become increasing apparent by reference to the following description and the drawings.

### SUMMARY

In the present disclosure, concentrated ammonia is used to prepare mixtures of lignocellulosic materials and grain (i.e., mixtures of cellulose, hemicellulose, and starch) for subsequent conversion to sugars by pretreating the mixed biomass at moderate temperatures and pressures. After removing most of the ammonia, the treated biomass is hydrolyzed by a combination of enzymes to simple sugars such as glucose and xylose. Using low levels (about 5 milligrams per gram of cellulose) of enzymes, essentially 100% of the cellulose and starch can be converted to glucose and over 85% of the xylan to xylose. Furthermore, the value of these mixed biomass streams (starch plus cellulose) as animal feeds is greatly enhanced. Process economic modeling shows that this approach to pretreatment based on ammonia is more cost effective than any other currently available and well studied approach.

Many crops produce both a starchy fraction (the grain) and also a cellulose-rich straw or crop residue such as corn stover, rice straw, or wheat straw. The disclosed processes preferably involve the treatment of mixed grains and cellulosic materials, for example corn and corn stover that have been harvested in a single pass through the field, with ammonia to increase the subsequent enzymatic conversion of the starch and cellulosic portions to fermentable sugars or digestible animal feedstuffs. Development of highly digestible animal feeds or fermentation feedstocks from mixed starchy/cellulosic materials improves the economics of both animal feeding and fermentation industries based on starch and cellulose and can provide environmental benefits as well. Preferably, the disclosure relates to the treatment of cellulose fiber rich streams from corn processing (e.g., distillers grains with solubles (DDGS) and/or corn fiber) which contain both cellulose and starch, to improve the extraction of the starch and/or the conversion of starch, cellulose and hemicellulose to sugars by enzymes.

Sufficiently inexpensive sugars from renewable plant biomass can become the basis of a very large chemical and fuels industry, replacing or substituting for petroleum and other fossil feedstocks. Effective and economical pretreatments are required to make these sugars available at high yield and acceptable cost. The present disclosure fills this requirement by providing an economical and effective pretreatment for lignocellulosic materials. Furthermore, this disclosure improves on existing pretreatment approaches by also being capable of enhancing the value of mixed grain (starchy material) and lignocellulose-containing feedstocks such as a harvested whole corn plant or the whole wheat plant. Previously processed fiber-rich and starch-containing materials such as corn fiber can also be advantageously treated by the process.

In an embodiment, a process for producing sugars from plant biomass comprises: (a) providing a comminuted plant biomass comprising cellulose, hemicellulose, and starch; (b) treating the comminuted plant biomass with concentrated ammonia under pressure in a closed vessel and then relieving the pressure to provide a treated plant biomass and to release (and preferably recover/recycle) the ammonia; and (c) hydrolyzing the treated plant biomass in the presence of water to form sugars using a combination of enzymes which hydrolyze the cellulose, the hemicellulose, and the starch to produce the sugars, the enzymes comprising an amylase and a cellulase (and optionally a hemicellulase). Preferably, the plant biomass is selected from corn (e.g., corn silage), wheat, and/or rice. The plant biomass can be a whole plant comprising an edible grain of the plant and a lignocellulosic portion of the plant, the edible grain and the lignocellulosic portion of the plant having been harvested at the same time. Preferably, process operating conditions include: (1) a water content in step (c) between about 0.1 kg and 2 kg water/kg of dry plant biomass, (2) an ammonia content in step (b) between about 0.2 kg and 2 kg of ammonia/kg of dry plant biomass, and/or (3) a temperature in the closed vessel in step (b) ranges from about 50° C. and 150° C. Preferably, step (b) of the process further comprises: (b-1) maintaining the closed vessel at a preselected temperature for a preselected time and an elevated pressure ranging from about 100 psi to 500 psi, and then (b-2) explosively releasing the pressure from the closed vessel, thereby causing disruption of the biomass by the ammonia. The hydrolyzed plant biomass can be further processed by filtering the water from the hydrolyzed plant biomass to separate and recover the formed sugars and/or by fermenting the sugars to form ethanol.

In another embodiment, a process for producing a fermentation product from a whole-plant biomass processed as a unit comprises: (a) providing a monocot whole-plant biomass comprising an edible grain of the plant biomass and a lignocellulosic portion of the plant biomass, the edible grain and the lignocellulosic portion of the plant biomass having been harvested at the same time; (b) treating the monocot whole-plant biomass with ammonia under pressure and then rapidly releasing the pressure to provide a disrupted cellulosic plant biomass; (c) hydrolyzing the disrupted cellulosic plant biomass to form sugars using a combination of enzymes comprising at least an amylase and a cellulase; and (d) fermenting the formed sugars to produce a fermentation product (e.g., ethanol). Preferably, step (b) comprises performing an Ammonia Fiber Explosion (Expansion) process (AFEX). The monocot whole-plant biomass can be soaked in water or a dilute combination of water and ammonia for a period of time prior to the treatment in step (b) to enhance sugar production.

In another embodiment, a process for producing sugars from whole-plant biomass comprises: (a) providing a comminuted whole-plant biomass selected from the group consisting of corn, wheat, rice, and combinations thereof, the whole-plant biomass comprising an edible grain of the plant biomass and a lignocellulosic portion of the plant biomass, the edible grain and the lignocellulosic portion of the plant biomass having been harvested at the same time; (b) treating the whole-plant biomass with about 0.2 kg to 2 kg of ammonia/kg of dry plant biomass in a closed vessel at an elevated pressure ranging from about 100 psi to 200 psi and an elevated temperature ranging from about 50° C. to 100° C. for a preselected time, and then explosively releasing the pressure to provide a disrupted cellulosic plant biomass; (c) hydrolyzing the disrupted cellulosic plant biomass to form sugars with enzymes comprising (i) an amylase to hydrolyze starch in the treated whole-plant biomass and (ii) a cellulase (and an optional hemicellulase) to hydrolyze cell wall components in the treated whole-plant biomass, thereby producing the sugars.

In a refinement of the foregoing processes, the combination of enzymes comprises a first enzyme and a second enzyme, and step (c) of the processes further comprises: (c-1) adding the first enzyme to the treated plant biomass and allowing the first enzyme to hydrolyze the treated plant biomass for a first preselected time, and then (c-2) adding the second enzyme to the treated plant biomass and allowing the second enzyme to hydrolyze the treated plant biomass for a second preselected time. Preferably, the first enzyme comprises an amylase to hydrolyze starch in the treated plant biomass and the second enzyme comprises a cellulase to hydrolyze cell wall components in the treated plant biomass. In various embodiments, the first enzyme either is or is not deactivated prior to adding the second enzyme to the treated plant biomass. In another refinement, the combination of enzymes is selected such that (i) the amylase comprises one or more of α-amylase and glucoamylase; (ii) the cellulase comprises one or more of endocellulase, exocellulase, and β-glucosidase; and (iii) the hemicellulase comprises one or more of xyloglucanase, β-xylosidase, endoxylanase, α-L-arabinofuranosidase, α-glucuronidase, and acetyl xylan esterase. Preferably, the combination of enzymes comprises about 1 mg amylase/g glucan to about 100 mg amylase/g glucan and/or about 1 mg cellulase (or a cellulase/hemicellulase mixture)/g glucan to about 100 mg cellulase (or a cellulase/hemicellulase mixture)/g glucan.

All patents, patent applications, government publications, government regulations, and literature references cited in this specification are hereby incorporated herein by reference in their entirety. In case of conflict, the present description, including definitions, will control.

Also related are Provisional Application No. 60/936,509, filed Jun. 20, 2007; PCT Application Nos. PCT/US07/10410, filed Apr. 30, 2007 (WO 2008/020901) and PCT/US07/10415, filed Apr. 30, 2007 (WO 2007/130337); U.S. application Ser. No. 11/729,632, filed Mar. 29, 2007; Dale et al. U.S. patent application entitled “Process for Enzymatically Converting a Plant Biomass” and filed Aug. 8, 2008; U.S. Pat. No. 6,106,888 to Dale et al. and U.S. Pat. No. 6,176,176 to Dale et al., which are incorporated herein by reference in their entireties.

Additional features of the disclosure may become apparent to those skilled in the art from a review of the following detailed description, taken in conjunction with the drawings, examples, and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a more complete understanding of the disclosure, reference should be made to the following detailed description and accompanying drawings wherein:

FIGS. 1A to 1D show a flow chart of details about old approaches of FIGS. 1A and 1C and beside new approaches as seen in FIGS. 1B and 1D to make ethanol. FIG. 1B shows the whole plant approach and FIG. 1D shows the whole grain approach.

FIGS. 2A and 2B are pictures showing untreated and AFEX treated materials. FIG. 2A shows whole plant (grain+stover) and FIG. 2B shows whole grain. The AFEX treated material is destructured.

FIG. 3 is a graph showing a comparison of enzymatic hydrolysis yields of glucose as a function of time for whole grains with and without AFEX pretreatment for corn, wheat and rice. All samples contained approximately 100 mg amylase/g glucan and 15 FPU (filter paper units)/g glucan cellulase, and were incubated at 50° C. and 90 RPM stirring. Yields are given after both 12 and 72 hours of hydrolysis.

FIG. 4 is a graph showing the effect of cellulase loading and deactivation of amylase enzymes prior to adding the cellulase on glucose yields for AFEX treated corn grain. Cellulase was added 12 hours after adding 100 mg amylase/g glucan.

FIG. 5 is a graph showing Whole Corn Plant (WCP) glucan hydrolysis yields for untreated and AFEX treated WCP after adding enzymes (amylase and cellulase) at the start of the reaction as a function of time. (Unt: Untreated, C: cellulase, βG: β-glucosidase, S: STARGEN amylase). Total concentration of enzymes loaded were 15 FPU/g glucan (˜33 mg protein/g glucan) of cellulase (SPEZYME CP, Genencor, a division of Danisco, Copenhagen, Denmark), 64 p-NPGU (p-nitrophenyl glycoside units)/g glucan (˜38 mg protein/g glucan) of β-glucosidase (NOVOZYM 188, Novozyme, Bagsvaerd, Denmark) and 10/50/100 microlitres (for 15 ml total reaction volume, ˜10/50/100 mg protein/g glucan) of amylases (STARGEN, Genencor).

FIG. 6 is a graph showing the effects of sequential addition of enzymes (amylase followed by cellulase and βG) on Whole Corn Plant (WCP) and glucan hydrolysis yields. (Unt: Untreated, C: cellulase, βG: β-glucosidase, S: STARGEN amylase). Total concentration of enzymes loaded were 15 FPU/g glucan (˜33 mg protein/g glucan) of cellulase (SPEZYME CP, Genencor, a division of Danisco, Copenhagen, Denmark), 64 p-NPGU/g glucan (˜38 mg protein/g glucan) of β-glucosidase (NOVOZYM 188, Novozyme, Bagsvaerd, Denmark) and 10/50/100 μl (for 15 ml total reaction volume, ˜10/50/100 mg protein/g glucan) of amylases (STARGEN, Genencor).

FIG. 7 is a graph showing sequential addition of enzymes (amylase followed by cellulase and βG) and glucan hydrolysis yields for AFEX treated and untreated whole plants. (Unt: Untreated, WCP: Whole Corn Plant, WRP: Whole Rice Plant, WWP: Whole Wheat Plant).

FIG. 8 is a graph showing the sugar yield both for untreated (UT) and AFEX-treated whole plant (C: whole corn plant; W: whole wheat plant; and R: whole rice plant). Commercial enzymes (5 mg/g of STARGEN amylase and 5 mg/g of ACCELERASE cellulase per g of glucan) were used during enzymatic hydrolysis.

FIG. 9 is a graph showing the sugar yield both for untreated (UT) and AFEX-treated corn silage. Both low (5 mg/g of STARGEN amylase and 5 mg/g of ACCELERASE cellulase per g of glucan) and high (H; 5 mg/g of STARGEN amylase and 10 mg/g of ACCELERASE cellulase per g of glucan) concentrations of commercial enzyme mixtures were used during enzymatic hydrolysis.

FIG. 10 is a graph showing the enzymatic conversion both for untreated (UT) and AFEX-treated samples of cellulose, starch, and a cellulose/starch mixture. Commercial enzymes (7.53 mg/g of STARGEN amylase and/or 10.73 mg/g of ACCELERASE cellulase per g of glucan) were used during enzymatic hydrolysis.

While the disclosed compositions and methods are susceptible of embodiments in various forms, specific embodiments of the disclosure are illustrated in the drawings (and will hereafter be described) with the understanding that the disclosure is intended to be illustrative, and is not intended to limit the claims to the specific embodiments described and illustrated herein.

### DETAILED DESCRIPTION

The specific features of this disclosure that make it more advantageous than old methods are as follows: (1) it does not degrade any biomass carbohydrates so that yield is not compromised due to the pretreatment; (2) relative to theoretically possible yields, high overall yields of glucose (e.g., at least about 90% or about 95% to 100% for corn, at least about 70% or about 70% to 80% for rice and wheat) and xylose can be obtained; (3) low application rates of otherwise expensive hydrolytic enzymes are needed to obtain these yields; (4) residual ammonia can serve as a nitrogen source for subsequent fermentations or animal feeding operations; (5) treated biomass and sugars can be fed at very high solids levels to subsequent process operations, thereby increasing the concentration of all products and reducing the expense of producing other chemicals from these sugars; (6) using ammonia and concentrated ammonium hydroxide combinations fits well into the most likely recovery operations for ammonia; (7) multiple engineering design approaches are available to minimize the amount of ammonia in the gaseous phase and to thereby maximize the effectiveness of the pretreatment for a given ratio of biomass to ammonia; (8) mixed starch and cellulosic streams (e.g., corn stover and corn grain) can be treated and their value enhanced together without the expense of separating the two different materials; and (9) additional plant material can be harvested with a single pass through the field, further reducing the cost and increasing the ease of obtaining digestible sugars from plant biomass.

Markets that might use the disclosed processes include: (1) the U.S. chemical industry which is beginning to move away from petroleum as a source of chemical feedstocks and is interested in inexpensive sugars as platform chemicals for new, sustainable processes; (2) the fermentation industry, especially the fuel ethanol production industry, which is also interested in inexpensive sugars from plant biomass; and (3) the animal feed industry which is strongly affected by the cost of available carbohydrates/calories for making animal feeds of various kinds.

Plant material or “biomass” is composed of carbohydrates (starch, cellulose, hemicellulose and simple sugars), protein, lignin, lipids, pectin, minerals and a host of minor components. Cellulose is a structural component of the biomass (e.g., cell wall component) and is a crystalline β(1→4) glycosidic polysaccharide. Hemicellulose is also a structural component of the biomass (e.g., cell wall component), and is generally an amorphous polysaccharide of multiple sugar monomers including glucose, xylose, mannose, galactose, rhamnose, and arabinose. Starch is a digestible component of the biomass (e.g., interior grain component) and generally includes amylose (α(1→4) glycosidic polysaccharide) and/or amylopectin (α(1→4) and α(1→6) glycosidic polysaccharide). While not particularly limited, suitable examples of plant biomass include those with substantial amounts of cellulose, hemicellulose, and starch such as corn, wheat, and/or rice. The combination of the cellulose, hemicellulose, and starch in the biomass makes the biomass difficult to process as a single unit with conventional methods. The plant biomass can be a whole grain or a whole plant (e.g., including an edible grain of the plant and a lignocellulosic portion (i.e., including cellulose, hemicellulose, and lignin) of the plant, where both the edible grain and the lignocellulosic portion of the plant were harvested at the same time), and the processed plant biomass can be freshly harvested, stored for a short period (e.g., up to several days), or stored for an extended period (e.g., up to about a year, in the case of corn silage). Prior to further treatment, the biomass is preferably milled or otherwise comminuted by any conventional means (e.g., to a particle size ranging from about 100 μm to about 1000 μm, for example as specified in the NREL LAP protocol, NREL Technical Report NREL/TP-510-42620, incorporated herein by reference). Because of its low cost and abundance, biomass can potentially be a large scale source both of organic chemicals and fuels. To achieve this potential, it is necessary to “refine” biomass by breaking it down into its primary constituents. Biomass processing using ammonia and related technologies achieves a high degree of refining while avoiding many of the limitations and problems associated with other refining approaches.

In the disclosed processes, water and/or ammonia at various concentrations is used to effect a “refining” (or separation) of biomass into multiple product streams that can then be upgraded by further processing. Ammonia in water at lower ammonia concentrations (lower than saturation ammonia concentrations) and moderate temperatures is used to extract and remove soluble species such as simple sugars, soluble minerals and proteins from biomass and/or to enhance the overall economics of the complete pretreatment process. This stream of solubles is further processed to produce solid protein products for human and animal feeds as well as a residual liquid stream that might be used as a supplement for microbial fermentation or for further processing to multiple products.

Ammonia concentration and the system temperature are then increased on the extracted solids to obtain ammonia concentrations and temperatures requiring greater than ambient pressure to maintain the desired ammonia concentration at the particular temperature. Suitable ammonia concentration range from about 0.1 g/g to 4 g/g (alternatively about 0.2 to 2, about 0.2 to 1, or about 0.5 to 1; units: g ammonia/g dry plant biomass). Similarly, suitable temperatures range from about 50° C. to about 200° C. (alternatively about 50° C. to about 150° C., about 50° C. to about 100° C., or about 70° C. to about 90° C.); however, even relatively low temperatures ranging from about 50° C. to about 60° C. can be used. The pretreatment of plant biomass in a closed vessel reactor at such conditions causes the pressure in the vessel to increase (i.e., due to the heat-induced evaporation of water and ammonia into the ractor headspace). Generally, the generated pressure can range from about 100 psi to about 500 psi, although pressures ranging from about 100 psi to about 200 psi are often suitable. In an embodiment, the pressure in the reactor can selectively controlled with an external pressure control (e.g., by selectively releasing some gas prior to the explosive AFEX pressure release, by adjusting the interior volume of the reactor, and/or by adding an additional source gas to the reactor). When biomass is pretreated under appropriate sets of these above-ambient pressure conditions, the structural carbohydrates in biomass (cellulose and hemicellulose) become much more susceptible to hydrolysis by enzymes and acid. Following this pretreatment step at high ammonia concentrations, the pressure is released allowing much of the ammonia to evaporate and the system to cool. Additional ammonia can be removed by heating, stripping with inert gases, reducing the pressure and the like.

Following pressure release, another extraction of the solids with ammonium hydroxide solutions may be done to recover additional protein or to remove part of the lignin from the biomass. Ammonia is recovered from the liquid and gas phases of the different process steps and recycled. Adequate ammonia or ammonium salts are left behind in the liquid or solid phases to assist microbial growth in subsequent fermentation steps or to serve as an ingredient in animal feeds at appropriate levels.

After adjusting the pH and temperature, the resulting solids are hydrolyzed with enzymes to produce simple sugars and/or sugar oligomers having a desired molecular weight distribution. The precise set of enzymatic activities used is tailored to generate the sugars (e.g., glucose, xylose, arabinose, mannose, etc. and their oligomers) at the desired concentrations and yields. Conventional cellulase mixtures used to hydrolyze acid-treated biomass may not be completely adequate for conversion of ammonia-treated biomass since ammonia does not generate monomeric sugars as does acid. Proper hemicellulase activities can be used for hydrolysis of ammonia-treated biomass. Hydrolyzed solids containing adsorbed enzymes can be contacted with fresh, unhydrolyzed solids to recover and reuse a portion of the enzyme.

Preferably, the AFEX-treated biomass is hydrolyzed with a combination of enzymes which hydrolyze the cellulose, the hemicellulose, and the starch to produce the sugars. Suitable combinations include (1) an amylase, (2) a cellulase, and (3) preferably a hemicellulase. The amylase includes one or more enzymes that hydrolyze starch (as both amylose and amylopectin) to form simpler sugars, ultimately yielding glucose monosaccharides, for example α-amylase (endoamylase) and/or glucoamylase (exoamylase). The cellulase includes one or more enzymes that hydrolyze cellulose to form simpler sugars, also ultimately yielding glucose monosaccharides, for example endocellulase (endoglucanase), exocellulase (exoglucanase), and/or β-glucosidase (cellobiase). The hemicellulase includes one or more enzymes that hydrolyze hemicellulose to form simpler sugars, ultimately yielding monosaccharides (e.g., glucose, other hexoses, pentoses). Suitable hemicellulases include one or more of xyloglucanase, β-xylosidase, endoxylanase, α-L-arabinofuranosidase, α-glucuronidase, and acetyl xylan esterase. Preferably, the enzymes include a combination of both endo-enzymes (i.e., enzymes hydrolyzing internal polysaccharide bonds to form smaller poly- and oligosaccharides) and exo-enzymes (i.e., enzymes hydrolyzing terminal and/or near-terminal polysaccharide bonds to form mono-, di-, tri-, tetra-, etc. saccharides) to facilitate both the rapid hydrolysis of large polysaccharide molecules and the formation of monosaccharide glucose products. A suitable commercial amylase mixture is STARGEN (available from Genencor, a division of Danisco, Copenhagen, Denmark) containing α-amylase and glucoamylase. A suitable commercial cellulase/hemicellulase mixture is SPEZYME (Genencor/Danisco) containing endoglucanase, exoglucanase, β-glucosidase, and hemicellulases (including xyloglucanase, β-xylosidase, endoxylanase, α-L-arabinofuranosidase, and α-glucuronidase). Another suitable commercial cellulase/hemicellulase mixture is ACCELERASE (Genencor/Danisco) containing endoglucanase, exoglucanase, β-glucosidase, and hemicellulase. A suitable commercial β-glucosidase is NOVOZYM (available from Novozyme, Bagsvaerd, Denmark) containing primarily β-glucosidase and minor amounts of endoglucanase and exoglucanase.

In an embodiment, the combination of enzymes is added sequentially to the AFEX-treated biomass: (1) a first enzyme of the combination is added to the biomass and allowed to hydrolyze the biomass for a first preselected time, and then (2) a second enzyme of the combination is added to the biomass and allowed to hydrolyze the biomass for a second preselected time. In an embodiment, the first enzyme is an amylase that hydrolyzes starch in the biomass (e.g., for about 12 hr) and the second enzyme is a cellulase that hydrolyzes cell wall components in the biomass (e.g., for an additional time of about 12 hr or more). In another embodiment, however, the first enzyme can be the cellulase and the second enzyme can be the amylase. An advantage of such a sequential-addition scheme for the enzymes is that it does not require a deactivation step prior to adding the second enzyme (i.e., the presence of the first enzyme does not substantially reduce the activity of the second enzyme such that the first enzyme must be deactivated before any hydrolytic activity from the second enzyme can be achieved). However, a deactivation step can be used and can improve sugar conversion in some cases (e.g., in particular when a whole plant biomass is used). The sequential-addition scheme also can improve sugar conversion.

The enzymes generally can be used in amounts that are not particularly limited. For example, amylase, cellulase, hemicellulase, or cellulase/hemicellulase mixtures individually can be used in amounts ranging from about 0.1 mg/g to about 500 mg/g (e.g., about 0.5 mg/g to about 200 mg/g, about 1 mg/g to about 100 mg/g, about 2 mg/g to about 50 mg/g, or about 3 mg/g to about 40 mg/g). The concentration units are milligrams of combined enzymes (e.g., combined amount of α-amylase and glucoamylase in an amylase mixture) per gram of total glucan in the plant biomass (i.e., glucose derivable from starch, cellulose, and a portion of hemicellulose). However, an advantage of the disclosed process is that the AFEX pretreatment permits the effective use of relatively low concentrations of otherwise expensive hydrolytic enzymes. For example, low-concentration amylase, cellulase, hemicellulase, or cellulase/hemicellulase mixtures individually can be used in amounts ranging from about 1 mg/g to about 20 mg/g, about 2 mg/g to about 15 mg/g, or about 3 mg/g to about 12 mg/g.

An example of a suitable processing sequence includes:

(1) Add hot ammonium hydroxide/water solutions or hot ammonia/water vapors to ground mixtures of lignocellulose and starchy grains (called hereinafter “mixed biomass”) in contained environments to obtain final mixture temperatures ranging from about 50° C. to 200° C.

(2) Obtain an intermediate ammonia content ratio of about 0.1 to 4 (alternatively about 0.2 to 2, about 0.2 to 1, or about 0.5 to 1; units: g ammonia/g dry plant biomass) and an intermediate water content ratio of about 0.1 to 4 (alternatively about 0.2 to 2, about 0.2 to 1, or about 0.4 to 1; units: g water/g dry plant biomass).

(3) Allow sufficient time for reaction to occur under these conditions, approximately 5 minutes (e.g., about 1 min to 20 min or about 2 min to 10 min).

(4) Compress the ammonia-treated mixed biomass, for example, in a screw reactor, to minimize the volume of vapor or “dead” space.

(5) Further reduce the tendency of ammonia to convert to a gas by, for example, pressurizing the system with an inert gas such as nitrogen, or by mixing finely divided solids such as sand or iron filings with the mixed biomass, or by connecting the headspace of the treatment vessel with the headspace of the ammonia or ammonia:water supply vessel.

(6) Add highly concentrated ammonia:water mixtures to the intermediate combination of mixed biomass, ammonia and water to obtain a final ammonia level of about 0.5 g ammonia (as NH₃) per g of dry biomass (e.g., about 0.1 to 4, about 0.2 to 2, about 0.2 to 1, or about 0.5 to 1) and temperatures up to about 90° C. (e.g., about 50° C. to 200° C.).

(7) Hold new mixture at these conditions for about an additional 5 minutes (e.g., about 1 min to 20 min or about 2 min to 10 min).

(8) Rapidly release the pressure to remove and preferably recover/recycle the ammonia.

(9) Hydrolyze the resulting solids with a combination of enzymes to mixtures of simple sugars containing, for example, glucose, xylose and arabinose; and

(10) Feed the treated biomass to appropriate animal species or use it as a fermentation feed.

### EXAMPLES

The following examples illustrate the disclosed compositions and methods, but are not intended to limit the scope of any claims thereto.

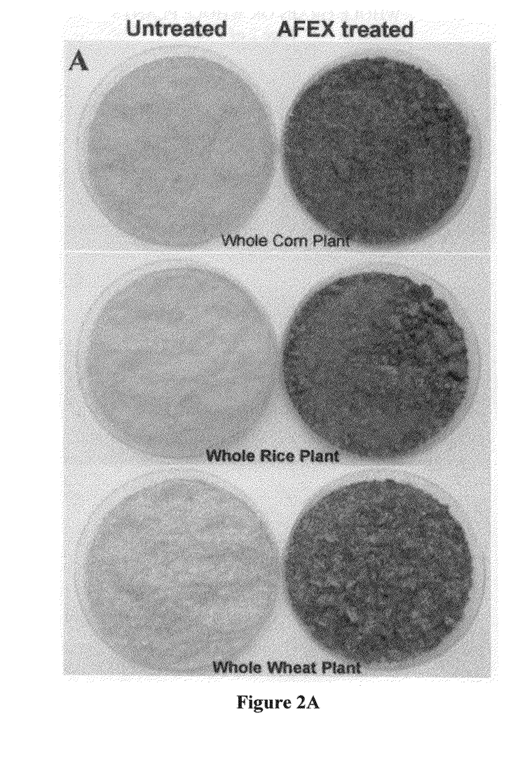

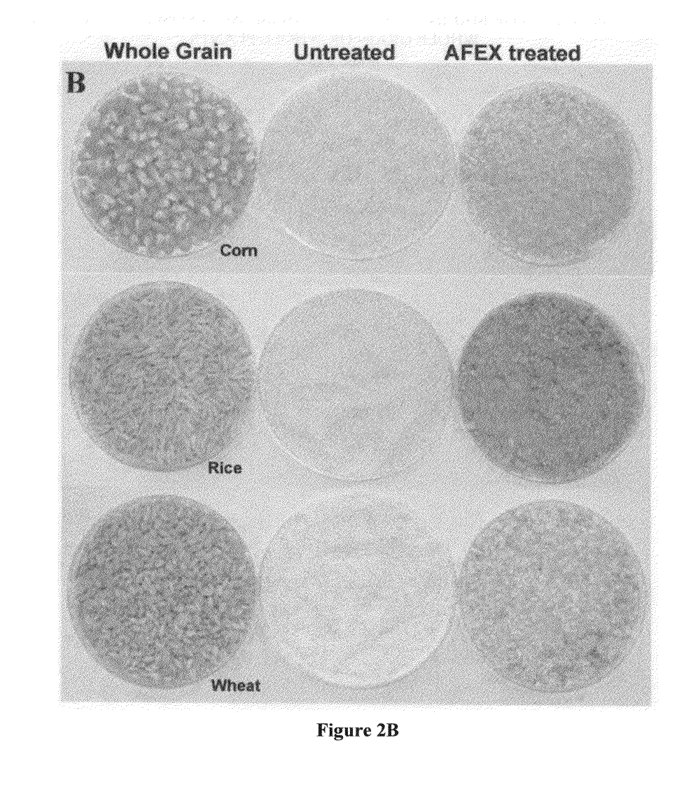

AFEX Pretreatment: Plant biomass (e.g., whole grain or whole plant) was pretreated by an AFEX pretreatment process. Unless otherwise stated, the AFEX pretreatment process was performed according to the following general conditions. The biomass with 60% moisture (kg water/kg dry biomass) was transferred to a high-pressure Parr closed vessel reactor and liquid anhydrous ammonia (1 kg of ammonia/kg of dry biomass) was slowly charged to the vessel. The temperature was raised and maintained in the range of 50° C. to 90° C. for a 5-minute residence time at the selected temperature before explosively relieving the pressure. About 20-25 minutes elapsed while the biomass was heated and ammonia was added to the reaction vessel; the residence time of about 5 minutes represents an additional constant-temperature hold time once the desired temperature was reached (i.e., a preselected temperature in the range of 50° C. to 90° C.), resulting in a net AFEX-pretreatment time of about 25-30 minutes. Throughout the pretreatment process, the increasing temperature caused water and ammonia to vaporize, filling the reactor headspace and pressurizing the reactor to a pressure of about 150 psi (generally in the range of about 100 psi to about 200 psi). Once released, the instantaneous drop of pressure in the vessel caused the ammonia to vaporize, releasing the ammonia and resulting in an explosive decompression of the biomass and considerable biomass fiber disruption. The AFEX-pretreated material was allowed to stand under a hood overnight to remove the residual ammonia and was stored in a freezer until further use. Untreated plant biomass was also used as a control for the various examples. Pictures of untreated and AFEX-treated whole plant and whole grain materials are given in FIGS. 2A (whole plant) and 2B (whole grain).

Enzymatic Hydrolysis and Sugar Analysis: The NREL standard protocol (LAP-009) was followed for enzymatic hydrolysis of the biomass. The substrate (corn stover and Avicel (microcrystalline cellulose)) was hydrolyzed at a glucan loading of 1% (w:v) in a 0.05 molar citrate buffer solution (pH 4.8) at a desired cellulase enzyme (SPEZYME CP; Genencor International, Rochester, N.Y.) loading (protein concentration 123 mg/ml) of up to 15 FPU/g glucan (˜33 mg protein/g glucan) and β-glucosidase enzyme (Sigma, St. Louis, Mo.) loading of 64 pNPGU/g glucan (˜38 mg protein/g glucan), and/or 10 to 100 microliters (˜10-100 mg protein/g glucan) amylase enzyme (STARGEN; Genencor). In some examples, an enzyme combination including a STARGEN amylase loading of 5 mg/g glucan and an ACCELERASE cellulase (Genencor International, Rochester, N.Y.) loading of 5-10 mg/g glucan was used. A High Performance Liquid Chromatography (HPLC) system was used for sugar analysis.

Summary of Experimental Conditions: The processing conditions (plant biomass, AFEX parameters, and hydrolysis parameters) for Examples 1-7 are summarized in Table 1. Additional details for each example are presented below.

TABLE 1
Summary of Processing Conditions

| | Example | | | | | | |
|---|---|---|---|---|---|---|---|
| | 1 | 2 | 3 | 4 | 5 | 6 | 7 |
| Biomass | | | | | | | |
| Type | Whole | Whole | Whole | Whole | Whole | Whole | Whole Plant |
| | Grain | Grain | Plant | Plant | Plant | Plant | Silage |
| Plant | C, R, W | C | C | C | C, R, W | C, R, W | C |
| AFEX | | | | | | | |
| Temp. | 50° C. | 50° C. | 90° C. | 70-90° C. | 70-90° C. | 90° C. | 50° C. |
| NH₃ | 1.0 g/g | 1.0 g/g | 1.0 g/g | 0.5-1.0 g/g | 1.0 g/g | 1.0 g/g | 1.0 g/g |
| H₂O | 0.6 g/g | 0.6 g/g | 0.6 g/g | 0.0-0.6 g/g | 0.6 g/g | 0.6 g/g | 2.0 g/g |
| Time | 5 min | 5 min | 5 min | 5 min | 5 min | 5 min | 5 min |
| Hydrolysis | | | | | | | |
| Amylase | 100 mg/g S | 100 mg/g S | 10-100 μl S | 10-100 μl S | 50 μl S | 5 mg/g S | 5 mg/g S |
| Cellulase | 15 FPU C | 0-15 FPU C | 15 FPU C | 15 FPU C | 15 FPU C | 5 mg/g A | 5-10 mg/g A |
| | | | 64 pNPGU B | 64 pNPGU B | 64 pNPGU B | | |
| Seq. Add. | No | Yes | No | Yes | Yes | No | No |
| Time | 12-72 h | 48 h | 12-168 h | 12-168 h | 12-168 h | 24-72 h | 24-72 h |
| FIG. | 3 | 4 | 5 | 6 | 7 | 8 | 9 |

Notes:
(1) Plants are C: corn, R: rice, and W: wheat
(2) NH₃ and H₂O amounts are based on a dry plant biomass basis;
(3) Amylase enzyme is S: STARGEN;
(4) Cellulase enzymes are A: ACCELERASE B: β-glucosidase, and C: SPEZYME;
(5) “Seq. Add.” denotes the sequential addition of amylase prior to cellulase

### Examples 1 and 2

### Whole Grain Process

### Example 1

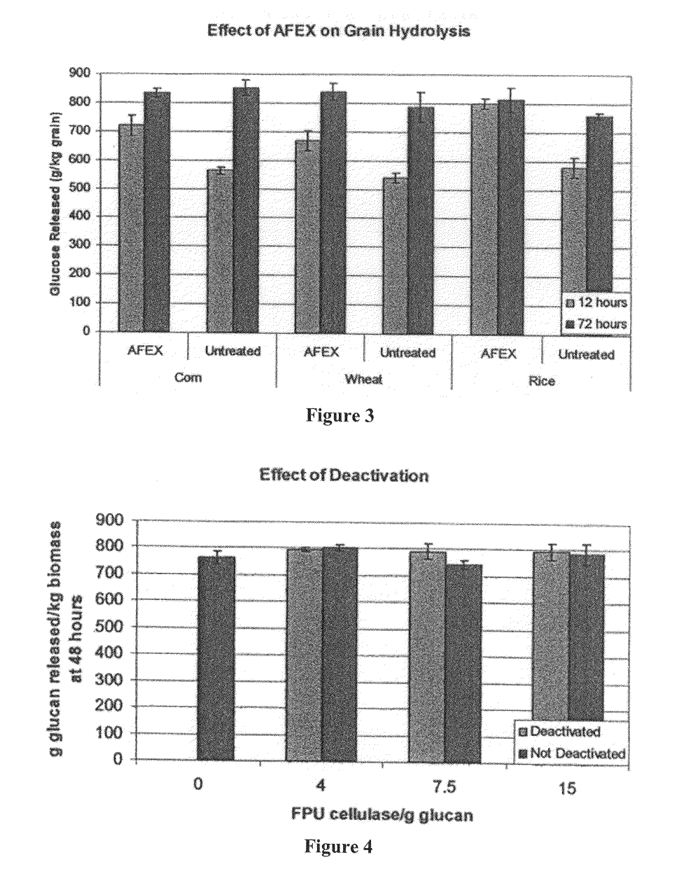

FIG. 3 compares the amount of glucose released during enzymatic hydrolysis between AFEX pretreated and untreated grain for three different grains. Yields are similar after 72 hours of hydrolysis for both corn and wheat, although yields appeared to significantly increase for rice grain. This may be due to the higher cellulose content in rice compared to the other two and thus, a greater portion of the overall glucan content is not susceptible to hydrolysis without the pretreatment. However, the initial rate of hydrolysis (based on 12 hours yield) is greater for all three grains with AFEX pretreatment than without. This strongly suggests that AFEX also makes starch more accessible to hydrolysis, as this difference can not be explained by the cellulose content alone. Thus, the hydrolysis time can be reduced if AFEX is used thereby improving process economics. This is most notable in the rice grain, as hydrolysis appears to be complete after only 12 hours, a six-fold reduction in residence time.

### Example 2

Amylase may inhibit the activity of cellulase enzymes, leading to non optimal enzyme loadings and yields. Furthermore, due to the low cellulose content in grains, it should be possible to lower the total enzyme loading, thus reducing the final cost. FIG. 4 shows the effect of different cellulase loadings on corn grain as well as the effect of deactivating the amylase prior to loading the cellulase. In all cases, amylase was added at the beginning of hydrolysis, while cellulase was added after 12 hours. The amylase was deactivated using heat coagulation/precipitation prior to adding the cellulase in some cases. Although deactivation improved yields at 7.5 FPU cellulase/g glucan (˜16 mg protein/g glucan), it did not appear to have an effect at either low or high enzyme loadings.

Furthermore, no decrease in overall yields is seen as cellulase loadings are decreased to 4 FPU/g glucan (˜9 mg protein/g glucan). These yields are higher than when no cellulase was added, indicating that the cellulose is being hydrolyzed in addition to the starch. Thus, it appears that only a small quantity of cellulase needs to be added to hydrolyze the fiber in grains, and that no potentially costly deactivation of previously added enzymes is required.

### Examples 3 to 6

### Whole Plant Process

Harvesting the whole plant, inclusive of grains and stalk, for ethanol bio-processing is an economical route for future biorefineries. Without a pretreatment, cellulosic whole plants would be much more difficult to hydrolyze completely than would starchy grains. With increasing cellulose content in the whole plant biomass, as shown in Table 2, we expect a much more recalcitrant cellulosic feedstock. It is also important to note that the biological source of the whole plant dictates its final composition (Table 2). Whole Corn Plant (WCP) has the highest starch content compared to Whole Rice (WRP) and Wheat Plants (WWP); in contrast, the cellulosic and hemicellulosic content of WWP is highest. These compositional differences within different species of whole plants would also have an important bearing on the pretreatment conditions and enzymatic hydrolysis results. We have compared the enzymatic hydrolysis glucan yields for three different whole plant crops, before and after AFEX pretreatment. We have also carried out a preliminary screening for the best AFEX conditions (Table 3; AFEX condition 8 later used for determining a suitable enzyme addition scheme for WCP).

TABLE 2
AFEX Whole Plant Glucan Composition

| Biomass Type | Total Glucan | Hemicellulose | Cellulose | Starch |
|---|---|---|---|---|
| Whole Corn Plant | 64.5 | 13.7 | 16.4 | 48.1 |
| (WCP) | | | | |
| Whole Rice Plant | 62.9 | 10.5 | 28.5 | 34.4 |
| (WRP) | | | | |
| Whole Wheat Plant | 59.3 | 23.3 | 34.0 | 25.3 |
| (WWP) | | | | |

TABLE 3
AFEX Preliminary Screening Conditions for Whole Plants

| | | Ammonia | |
|---|---|---|---|
| AFEX Type. | Temperature | Loading | Moisture % |
| 1 | 70° C. | 0.5 | n/a |
| 2 | 70° C. | 1 | n/a |
| 3 | 70° C. | 0.5 | 60 |
| 4 | 70° C. | 1 | 60 |
| 5 | 90° C. | 0.5 | n/a |
| 6 | 90° C. | 1 | n/a |
| 7 | 90° C. | 0.5 | 60 |
| 8 | 90° C. | 1 | 60 |
| 9 | 80° C. | 0.75 | 30 |

Note:
(1) Moisture “n/a” indicates no additional moisture added

### Examples 3 and 4

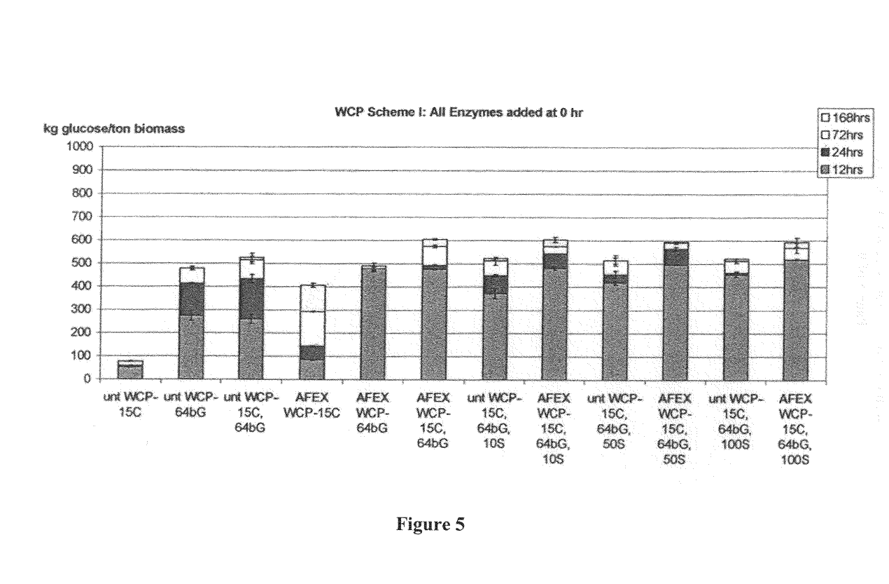

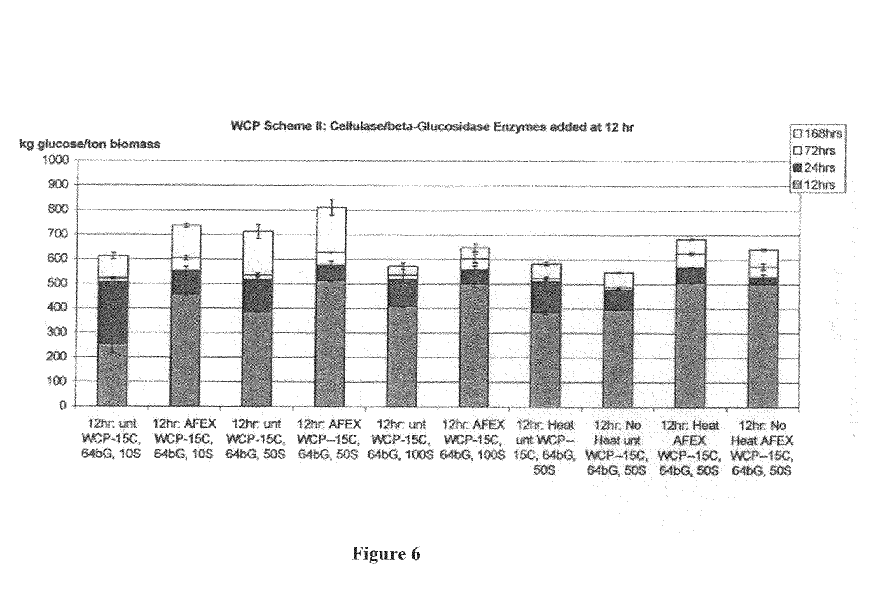

In order to screen the various AFEX conditions, it was necessary to first determine the optimal combination of enzymes and scheme of addition. A preliminary screening for the optimal combination and scheme of addition of enzymes was carried out for AFEX (90° C., 1:1, ammonia to biomass loading, 60% moisture, 5 minutes residence time) treated whole corn plant. The enzyme combination and scheme identified for AFEX-treated WCP was used for all subsequent hydrolysis. Amylolytic and cellulolytic enzyme systems are required for the complete hydrolysis of glucan in whole plants. However, due to antagonistic interaction between the two enzyme systems, finding the optimal enzyme concentrations and additional protocol is needed for complete glucan conversion. Hydrolytic data (FIG. 5) shows that adding all the enzymes together (cellulase and amylase) results in less synergistic hydrolysis results than adding the enzymes sequentially over a period of time (FIG. 6). The optimal enzyme addition scheme was found to be amylase addition (STARGEN) at 0 hr time period followed by cellulase addition (SPEZYME CP cellulase+NOVOZYM 188 β-glucosidase) at the end of 12 hrs of hydrolysis. It was also found that deactivating the amylase enzymes before addition of cellulolytic enzymes (denoted by “heat” in the caption of FIG. 6) resulted in slightly higher conversions for whole plants. In general, the observed improvement in conversion is more significant for whole plant biomass as compared to whole grain biomass, due to the fact that there is much more cellulose in the whole plant.

This trend of preferable sequential enzyme addition was not noticed for grains due to the higher cellulosic content of whole plants that presumably result in amylase-cellulase binding, preventing access to cellulolytic enzymes and hence, resulting in lower overall glucan conversions. The optimum STARGEN loading was 50 microliters per 15 ml reaction volume (at 1% glucan loading, ˜50 mg protein/g glucan) for 15 FPU/g glucan (˜33 mg protein/g glucan) cellulase loading and 64 p-NPGU/g glucan (˜38 mg protein/g glucan) based on hydrolysis of AFEX (90° C., 1:1, ammonia to biomass loading, 60% moisture, 5 minutes residence time) treated WCP. These enzyme loadings were used for all subsequent whole plant experiments; however, it is very likely a different enzyme loading might be appropriate for the whole rice and wheat plants due to higher cellulose and hemicellulose content.

### Example 5

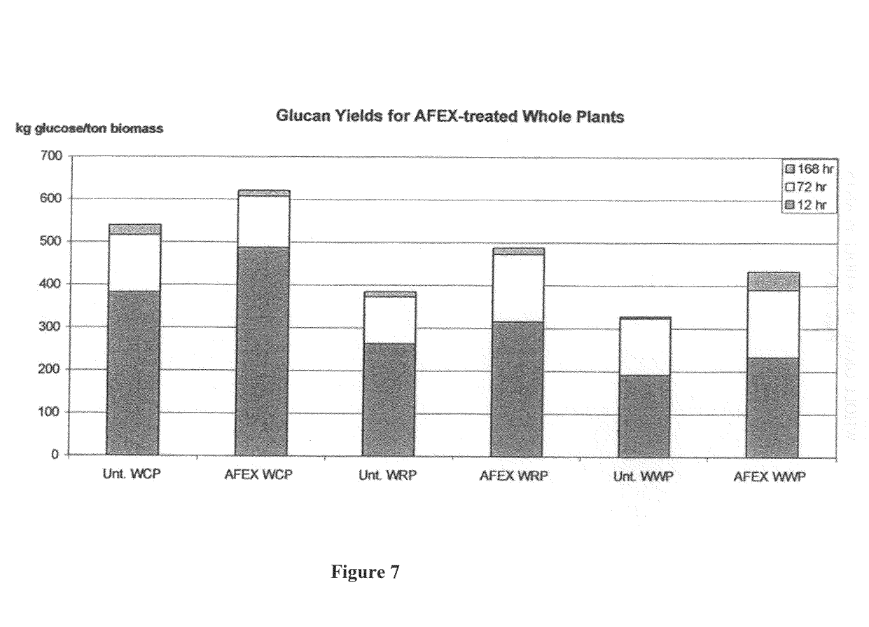

Using the identified enzyme loading profile from Examples 3 and 4 (i.e., 50 μl STARGEN, 15 FPU SPEZYME CP/g glucan, and 64 p-NPGU β-glucosidase/g glucan), the various AFEX conditions listed in Table 3 were tested. It was found that the optimum AFEX condition for whole corn plant (i.e., 70° C., 1:1 ammonia to biomass loading, 60% moisture, 5 minutes residence time) resulted in near complete glucan conversion. However, the best AFEX conditions found so far for whole rice and wheat plants was 90° C., 1:1 ammonia to biomass loading, 60% moisture, 5 minutes residence time. FIG. 7 illustrates the glucan yield for each of these three identified conditions. The maximum glucan yield for AFEX-treated whole corn plant was approximately 97% of the maximum possible (i.e., based on the total glucan content of Table 2). However, the maximum glucan yield was found to be 77% and 73% for AFEX-treated whole rice and wheat plant (also relative to the total glucan contents of Table 2), respectively, at the end of 168 hrs of hydrolysis. On the other hand, the untreated WCP, WRP and WWP had reached 83%, 61% and 55%, respectively, of maximum glucan yield at end of 168 hrs. The difference in glucan yields is slightly more pronounced at 12 and 72 hrs of hydrolysis. The data indicate an increased rate of hydrolysis and also the yield of hydrolysis as a result of the AFEX treatment for the whole plants. There can be further improvements in the hydrolytic yields of AFEX treated whole rice and wheat plants. An important observation made was that the higher the cellulosic and hemicellulosic content of the whole plant, the more severe the AFEX pretreatment required for complete glucan hydrolysis. The effect of AFEX on whole plants is more pronounced than that observed for grains alone due to the substantially higher lignocellulosic content. It might be a great advantage to the biorefinery process to simultaneously harvest the stalk and the grain for AFEX pretreatment and subsequent hydrolysis using the proper concoction of enzymes.

### Example 6

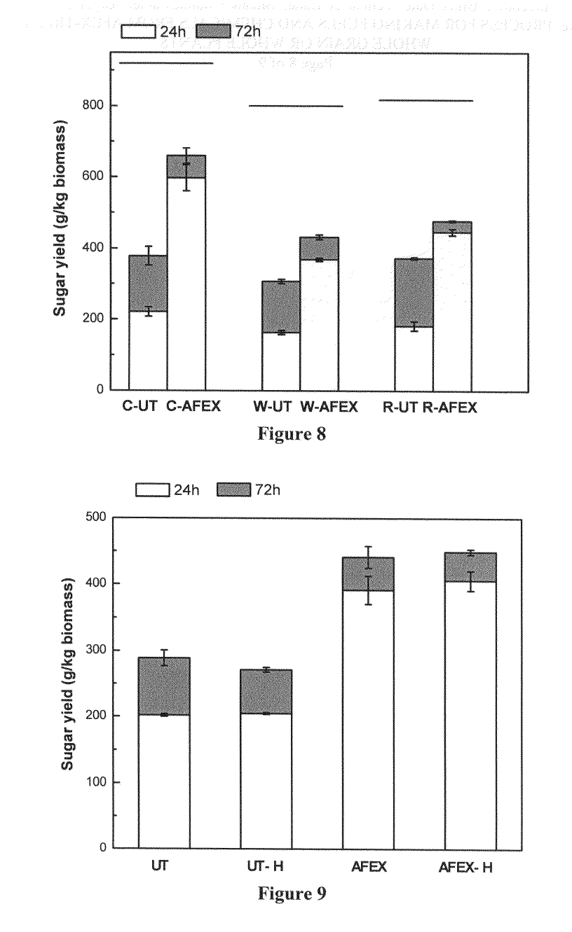

Example 6 illustrates the applicability of AFEX pretreatment to whole-plant biomass using different amylolytic and cellulolytic enzyme systems. In this case, an enzyme system using 5 mg/g of STARGEN amylase and 5 mg/g of ACCELERASE cellulase per g of glucan (both of which were added together) was used on AFEX-treated whole-plant biomass (corn, rice, and wheat). The AFEX conditions were: 90° C., 1:1 ammonia-to-biomass loading, 60% moisture, and 5 minutes residence time. FIG. 8 presents the results of Example 6, illustrating the sugar yield for both untreated and AFEX-treated plant biomass, with the solid overline representing the theoretical maximum sugar yield for the particular plant biomass. The AFEX pretreatment helps to open up the whole plant biomass and improves the enzymatic hydrolysis when compared to untreated whole plant. At a hydrolysis time of 24 hr, the AFEX treatment resulted in about 100%-200% higher sugar yield; at a hydrolysis time of 72 hr, the AFEX treatment still resulted in about 25%-40% higher sugar yield.

### Example 7

### Whole Plant Process (Corn Silage)

The disclosed process can be applied to whole-plant biomass in a state other than freshly harvested plant biomass (e.g., plant biomass that has been stored for a period of time prior to AFEX pretreatment). For example, a suitable whole-plant biomass includes corn silage—a whole corn plant that has been preserved by anaerobic bacterial fermentation. Corn silage is stored over a period of time, typically up to one year. During the fermentation process, the anerobic bacteria act on the plant biomass, resulting in a material that is rich in protein and can be suitably used as an animal feed. The longer the storage time available for fermentation, the composition of glucan (cellulose and starch) and xylan will be proportionately reduced decreased (i.e., the microbes consume the glucan and xylem, converting them to microbial biomass which is rich in protein). Table 4 summarizes some of the relevant components of corn silage as compared to corn stover (i.e., the stalk, leaf, husk, and cob of corn remaining following the harvest of corn for grain). From Table 4, the high ratio of cellulose to xylan in corn silage (i.e., about 5:1) makes corn silage a particularly attractive feed stock for the pretreatment of whole-plant biomass by AFEX.

TABLE 4
Comparison of Corn Stover and Corn Silage

| | Corn Stover | Corn Silage |
|---|---|---|
| Component | (wt. %) | (wt. %) |
| Cellulose | 34.1 | 49.1 |
| Xylan | 22.8 | 11.4 |
| Lignin | 11.4 | 8.8 |
| Protein | 2.3 | 10.2 |
| Fat | nd | 3.9 |

Example 7 illustrates the applicability of AFEX pretreatment to corn silage using an enzyme system of 5 mg/g of STARGEN amylase and 5 or 10 mg/g of ACCELERASE cellulase per g of glucan (both of which were added together). Similar to Table 3 above, a variety of AFEX conditions were screened to identify suitable parameters, with the ammonia loading ranging from about 0.1 g/g to about 3 g/g, the moisture content ranging from about 0.2 g/g to about 2 g/g, and the temperature ranging from about 50° C. to about 130° C. The identified AFEX conditions were: 50° C., 1:1 ammonia to biomass loading, 200% moisture, and 5 minutes residence time. FIG. 9 presents the results of Example 7, illustrating the sugar yield for both untreated and AFEX-treated plant biomass. The AFEX pretreatment helps to open up the corn silage biomass and improves the enzymatic hydrolysis when compared to untreated corn silage. At a hydrolysis time of 24 hr, the AFEX treatment resulted in about 100% higher sugar yield; at a hydrolysis time of 72 hr, the AFEX treatment still resulted in about 50% higher sugar yield.

### Example 8

### Enzymatic Hydrolysis of Cellulose and Starch Mixture

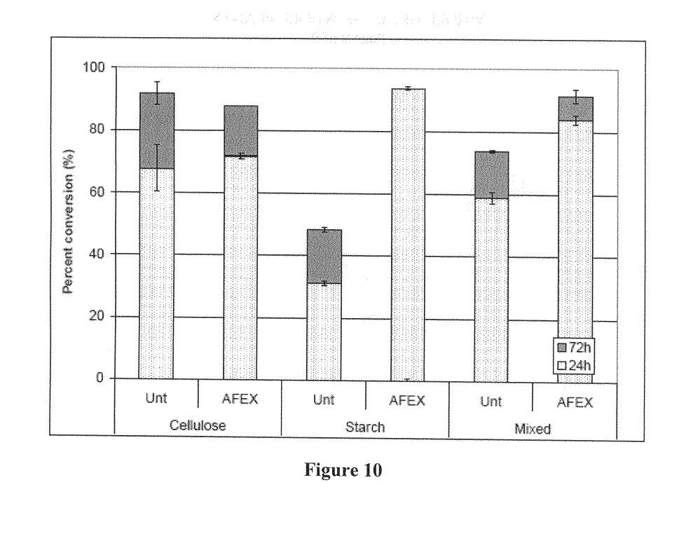

Example 8 illustrates the efficiency of AFEX pretreatment as applied to mixtures of cellulose and starch (i.e., a system representative of the glucan-containing materials found in plant biomass). FIG. 10 presents the enzymatic hydrolysis conversion on both untreated and AFEX-treated samples of cellulose alone (Avicel microcrystalline cellulose), starch alone, and a cellulose/starch mixture (50:50 wt. %). The AFEX conditions were: 90° C., 1:1 ammonia to biomass loading, 60% moisture, and 5 minutes residence time. The enzyme system used was 10.73 mg/g of ACCELERASE cellulase and/or 7.53 mg/g of STARGEN amylase per g of glucan. The cellulase and amylase enzymes were applied together in a mixture to the cellulose/starch mixture, but were used individually for the cellulose and starch samples. The primary insight of the results in FIG. 10 as applied to whole-plant hydrolysis rates and sugar yields is the effect AFEX treatment has on starch as well as the interactions between starch and cellulose hydrolysis. AFEX treatment results in dramatic improvements in pure starch hydrolysis as seen in FIG. 10 (e.g., about three times larger starch conversion at 24 hr for AFEX-treated vs. untreated starch). This is in contrast to the modest gains for pure cellulose. This is because, in lignocellulosic feedstocks, AFEX treatment primarily works on the hemicellulose and lignin, while doing little to solubilize or depolymerize lignin. Yet with starch, AFEX is likely solubilizing the starch prior to pressure release, dramatically increasing the rate and extent of digestion. This means that AFEX treatment can replace the steeping operation in traditional dry-grind ethanol plants without harming starch conversion.

The other major insight is the interaction when both starch and cellulose are present, as in the whole plant. In complex plant biomass mixtures (e.g., containing cellulose, hemicellulose, and starch), there is the potential that enzymatic systems for starch and cellulose hydrolysis can inhibit each other. For example, amylase enzymes may compete with cellulase enzymes by binding to a cellulose substrate, and/or vice versa. The results in FIG. 10 suggest that amylase-cellulase inhibition does not take place in the pristine cellulose/starch sample tested (i.e., the conversion at 24 hr for AFEX treatment of the mixed sample is roughly the average conversion of the individual cellulose and starch samples), likely a result of the pristine nature of the sample. If such competition/inhibition does occur in more complex plant biomass matrices, however, the hydrolytic conversion of the cellulose/starch mixture can be below what one would expect using a linear combination of the conversions as determined from cellulose-alone and starch-alone samples. However, because starch hydrolysis is so rapid with the AFEX treatment, high conversions can be nonetheless obtained using sequential enzyme addition coupled with intermediate enzyme deactivation, in particular for whole plant biomass. For example, the amylase enzymes are added first, allowing the majority of the starch to hydrolyze in a short period, and then the amylase enzymes are deactivated (e.g., through heating) prior to adding the additional cellulase enzymes. Suitable amylase hydrolysis times are up to about 24 hr, preferably up to about 12 hr, for example about 2 hr to about 24 hr, about 6 hr to about 18 hr, or about 12 hr. Suitable cellulase hydrolysis times are about 12 hr or more, preferably about 24 hr or more, for example about 12 hr to about 168 hr, about 24 hr to about 72 hr, or about 24 hr. Suitable heat deactivation can be performed at about 80° C. to about 150° C. (e.g., at about 100° C.) for about 5 min to about 120 min (e.g., about 20 min to about 40 min). Thus, both starch and cellulose are hydrolyzed in the same operation, eliminating potential drawbacks by achieving conversions larger than what would otherwise be expected when amylase-cellulase inhibitory interactions are present.

The processes according to the current disclosure can be contrasted with conventional processing methods as follows:

(A) Conventional Process: (1) the grains and stover are separated in the field; (2) the grains are then either wet-ground or dry-ground, and the resulting biomass is steam-cooked and converted to free sugars using amylase; (3) the pretreated lignocellulosic biomass from stover is separately converted to free sugars using cellulase and hemicellulase; and (4) the sugars generated by the above two separate processes can be combined or separately fermented to alcohol.

(B) Current Disclosure: (1) the whole grain or the whole plant (e.g., grains and stover) can be harvested together; (2) the resultant biomass pretreated using an AFEX process and hydrolyzed as a single unit to liberate sugars using different combinations of enzymes such as amylase, cellulase, and hemicellulase; (3) the sugars generated by the above processes can be fermented to alcohol (e.g., ethanol).

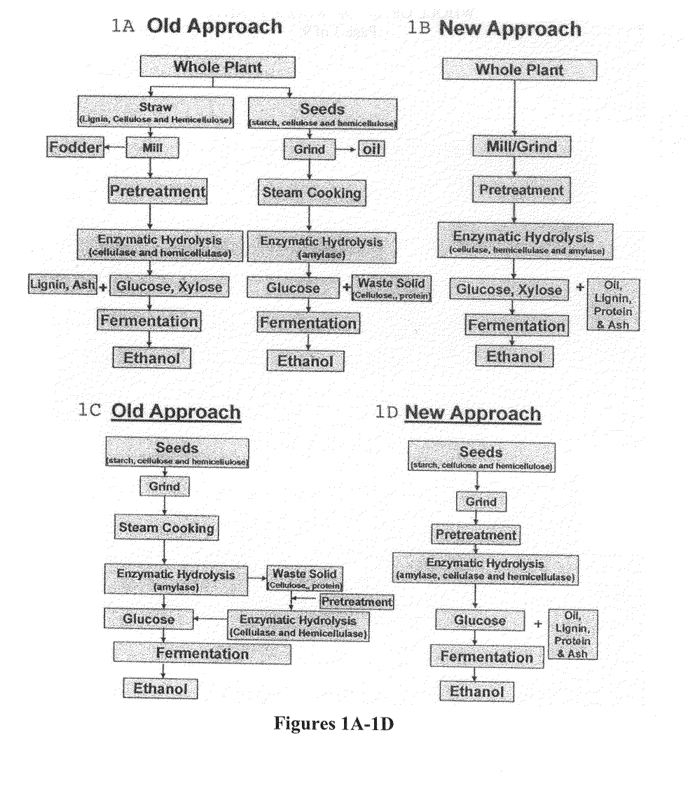

The conventional approach first separates grain and non-grain plant material so that the different plant materials can be treated in separate processes with methods and materials specific to the different plant materials (e.g., steam cooking with amylolytic hydrolysis of the seeds and a different pretreatment with cellulolytic hydrolysis of the non-seed material; as illustrated in FIG. 1A). Similarly, even when seeds alone are processed by conventional methods, two separate treatments and enzymatic hydrolysis steps are used (as illustrated in FIG. 1C). Thus, the disclosed process can eliminate multiple processing steps while retaining the benefits of high sugar yield attained by the multi-step methods: (1) the separation of grain and non-grain plant material and/or (2) the sequential application of multiple pretreatment and enzymatic hydrolysis steps. In addition to the streamlined processing, the disclosed process provides substantial advantages in terms of processing time and sugar yield. In general, the rate of sugar hydrolysis of AFEX-treated material is much more rapid than that of untreated material: at short hydrolysis times (e.g., up to about 24 hr, or about 12 hr to about 24 hr), substantially higher sugar yields are obtained for AFEX-treated material. Even at longer hydrolysis times (e.g., more than about 24 hr, or about 24 hr to about 168 hr), the relative yield of the untreated material increases; however, it is still generally at or below the yield of a similarly timed AFEX process.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

Throughout the specification, where the compositions, processes, or apparatus are described as including components, steps, or materials, it is contemplated that the compositions, processes, or apparatus can also comprise, consist essentially of, or consist of, any combination of the recited components or materials, unless described otherwise. Combinations of components are contemplated to include homogeneous and/or heterogeneous mixtures, as would be understood by a person of ordinary skill in the art in view of the foregoing disclosure.

### REFERENCES

1. Bothast, R. J. and Schlicher, M. A., Biotechnological processes for conversion of corn into ethanol, Appl. Microbiol Biotechnol., 67 (2005) 19-25.
2. Dale, B., “Method for increasing the reactivity and digestibility of cellulose with ammonia”, U.S. Pat. No. 4,600,590 (Jul. 15, 1986).
3. Dale, B. and Weaver, K., “Process for treating cellulosic materials”, U.S. Pat. No. 6,106,888 (Aug. 22, 2000).
4. Eggeman, T., and Elander, R. T., Process and economic analysis of pretreatment technologies, Bioresource Technol., 96 (2005) 2019-2025.
5. Energy Policy Act of 2005, sec. 1501, 109th Cong., 1st Sess., 2005.
6. From Niche to Nation: Ethanol Industry Outlook 2006; Renewable Fuels Association Washington D.C.: 2006, p 4.
7. Gray, K. A., Zhao, L., and Emptage, M., Bioethanol, Current Opinion in Chemical Biology, 10 (2006) 141-146.
8. Kamm, B., and Kamm, M., Principles of biorefineries, Appl Microbiol Biotechnol., 64 (2004) 137-145.
9. Knauf, M., and Moniruzzaman, M., Lignocellulosic biomass processing: A perspective, Int. Sugar J. 106 (2004) 147-150.
10. Mosier, N., Wyman, C., Dale, B., Elander, R. T., Lee, Y. Y., Holtzapple, M., and Ladisch, M., Features of promising technologies for pretreatment of lignocellulosic biomass, Bioresource Technol., 96 (2005) 673-686.
11. Ohara, H., Biorefinery, Appl. Microbiol Biotechnol., 62 (2003) 474-477.
12. Sun, Y., and Cheng, J., Hydrolysis of lignocellulosic materials for ethanol production: a review, Bioresource Technol., 83 (2002) 1-11.
13. Saha, B. C, Hemicellulose bioconversion, J. Ind Microbiol Biotechnol., 30 (2003) 279-291.
14. Sulbaran-de-Ferrer, B.; Aristiguieta, M.; Dale, B.; Ferrer, A.; Ojeda-de-Rodriguez, G. Appl. Biochem. Biotechnol. 105-108 (2003) 155-164.
15. Teymouri, F., Laureano-Perez, L., Alizadeh, H., and Dale, B. E., Optimization of the ammonia fiber explosion (AFEX) treatment parameters for enzymatic hydrolysis of corn stover, Bioresource Technol., 96 (2005) 2014-2018.
16. Zhang, Y-H. P., and Lynd, L. R., Toward an Aggregated Understanding of Enzymatic Hydrolysis of Cellulose: Non-complexed Cellulase Systems, Biotechnol. Bioeng., 88 (2004) 797-824.

What is claimed is:

1. A process for producing sugars from plant biomass, the process comprising:
(a) providing a comminuted plant biomass comprising cellulose, hemicellulose, and starch;
(b) treating the comminuted plant biomass with concentrated ammonia under pressure in a closed vessel and then relieving the pressure to provide a treated plant biomass and to release the ammonia; and
(c) hydrolyzing the treated plant biomass in the presence of water to form sugars using a combination of enzymes which hydrolyze the cellulose, the hemicellulose, and the starch to produce the sugars, the enzymes comprising an amylase and a cellulase.

2. The process of claim 1, wherein the plant biomass is selected from the group consisting of corn, wheat, rice, and combinations thereof.

3. The process of claim 2, wherein the plant biomass comprises corn silage.

4. The process of claim 1, wherein the plant biomass is a whole plant comprising an edible grain of the plant and a lignocellulosic portion of the plant, the edible grain and the lignocellulosic portion of the plant having been harvested at the same time.

5. The process of claim 1, wherein:
(i) the plant biomass has a water content in step (c) ranges from about 0.1 kg and 2 kg water/kg of dry plant biomass;
(ii) the plant biomass is contacted with the ammonia in step (b) in an amount ranging from about 0.2 kg and 2 kg of ammonia/kg of dry plant biomass; and
(iii) the temperature of the mixture of the ammonia and the plant biomass in the closed vessel in step (b) ranges from about 50° C. and 150° C.

6. The process of claim 1, wherein step (b) further comprises:
(b-1) maintaining the closed vessel at a preselected temperature for a preselected time and an elevated pressure ranging from about 100 psi to 500 psi, and then
(b-2) explosively releasing the pressure from the closed vessel, thereby causing disruption of the biomass by the ammonia.

7. The process of claim 1, further comprising:
(d) filtering the water from the hydrolyzed plant biomass to separate and recover the formed sugars.

8. The process of claim 1, wherein the enzymes in step (c) further comprise a hemicellulase.

9. The process of claim 8, wherein:
(i) the amylase comprises one or more of α-amylase and glucoamylase;
(ii) the cellulase comprises one or more of endocellulase, exocellulase, and β-glucosidase; and
(iii) the hemicellulase comprises one or more of xyloglucanase, β-xylosidase, endoxylanase, α-L-arabinofuranosidase, α-glucuronidase, and acetyl xylan esterase.

10. The process of claim 1, wherein the combination of enzymes comprises about 1 mg amylase/g glucan to about 100 mg amylase/g glucan and about 1 mg cellulase/g glucan to about 100 mg cellulase/g glucan.

11. The process of claim 1, wherein the combination of enzymes comprises a first enzyme and a second enzyme, and step (c) further comprises:
(c-1) adding the first enzyme to the treated plant biomass and allowing the first enzyme to hydrolyze the treated plant biomass for a first preselected time, and then
(c-2) adding the second enzyme to the treated plant biomass and allowing the second enzyme to hydrolyze the treated plant biomass for a second preselected time.

12. The process of claim 11, wherein the first enzyme comprises an amylase to hydrolyze starch in the treated plant biomass and the second enzyme comprises a cellulase to hydrolyze cell wall components in the treated plant biomass.

13. The process of claim 12, wherein the first enzyme is deactivated prior to adding the second enzyme to the treated plant biomass.

14. The process of claim 1, further comprising:
(d) fermenting the sugars, thereby forming ethanol.

15. A process for producing a fermentation product from a whole-plant biomass processed as a unit, the process comprising:
(a) providing a monocot whole-plant biomass comprising an edible grain of the plant biomass and a lignocellulosic portion of the plant biomass, the edible grain and the lignocellulosic portion of the plant biomass having been harvested at the same time;
(b) treating the monocot whole-plant biomass with ammonia under pressure and then rapidly releasing the pressure to provide a disrupted cellulosic plant biomass;
(c) hydrolyzing the disrupted cellulosic plant biomass to form sugars using a combination of enzymes comprising an amylase and a cellulase; and
(d) fermenting the formed sugars to produce a fermentation product.

16. The process of claim 15, wherein step (b) comprises performing an Ammonia Fiber Explosion (Expansion) process (AFEX).

17. The process of claim 15, wherein the monocot whole-plant biomass is soaked in water or a dilute combination of water and ammonia for a period of time prior to the treatment in step (b) to enhance sugar production.

18. The process of claim 15, wherein the fermentation product comprises ethanol.

19. The process of claim 15, wherein the enzymes in step (c) further comprise a hemicellulase.

20. A process for producing sugars from whole-plant biomass, the process comprising:
(a) providing a comminuted whole-plant biomass selected from the group consisting of corn, wheat, rice, and combinations thereof, the whole-plant biomass comprising an edible grain of the plant biomass and a lignocellulosic portion of the plant biomass, the edible grain and the lignocellulosic portion of the plant biomass having been harvested at the same time;
(b) treating the whole-plant biomass with about 0.2 kg to 2 kg of ammonia/kg of dry plant biomass in a closed vessel at an elevated pressure ranging from about 100 psi to 200 psi and an elevated temperature ranging from about 50° C. to 100° C. for a preselected time, and then explosively releasing the pressure to provide a disrupted cellulosic plant biomass;
(c) hydrolyzing the disrupted cellulosic plant biomass to form sugars with enzymes comprising (i) an amylase to hydrolyze starch in the treated whole-plant biomass and (ii) a cellulase to hydrolyze cell wall components in the treated whole-plant biomass, thereby producing the sugars.
